(19) United States
(12) Patent Application Publication
(10) Pub. No.: US 2026/0083333 A1
(43) Pub. Date: Mar. 26, 2026

(54) AUGMENTING PHYSIOLOGICAL SENSING BASED ON CONTACT CONDITION

(71) Applicant: Apple Inc., Cupertino, CA (US)

(72) Inventors: Ashkan OJAGHI, Milpitas, CA (US); Adriaan J. TAAL, San Diego, CA (US); Hany M. ARAFA, San Jose, CA (US); Imam uz ZAMAN, San Jose, CA (US)

(21) Appl. No.: 19/330,234

(22) Filed: Sep. 16, 2025

Related U.S. Application Data
(60) Provisional application No. 63/699,021, filed on Sep. 25, 2024.

(51) Int. Cl. A61B 5/01; A61B 5/00; G01K 1/02; G01K 3/14

(52) U.S. Cl. CPC A61B 5/01; A61B 5/681; A61B 5/6843; G01K 1/026; G01K 3/14; A61B 2560/0223; A61B 2562/0219; A61B 2562/0271

(57) ABSTRACT

A wearable device can include one or more temperatures sensors. Data from the one or more temperature sensors provide information, which can be used for the determination of the body temperature of a user wearing the electronic device. The accuracy of the determination of the body temperature of the user is dependent on variables such as the contact condition of the electronic device with the user. By determining the contact condition of the electronic device with the user, including measurements corresponding to one or more thermal emitters being in an activated state, the electronic device is optionally able to determine the contact condition and/or a correction factor, and thereby more accurately determine the temperature of the user.

### CROSS-REFERENCE TO RELATED APPLICATIONS

This application claims the benefit of U.S. Provisional Application No. 63/699,021, filed Sep. 25, 2024, the content of which is incorporated herein in its entirety for all purposes.

### FIELD OF THE DISCLOSURE

This relates generally to electronic devices including physiological sensing systems, such as temperature sensing systems, and more particularly to utilization of temperature data from temperature sensing systems of electronic devices for contact detection.

### BACKGROUND OF THE DISCLOSURE

An electronic device may include sensors, such as a touch sensor and an optical sensor. Another sensor that an electronic device may include is a temperature sensor.

### SUMMARY OF THE DISCLOSURE

This relates generally to systems and processes (e.g., algorithms, applications, and devices) that use temperature information for detecting the contact condition of a user-worn electronic device. Additionally or alternatively, this relates generally to systems and processes that use temperature information for further determining the temperature of a user using one or more temperature sensors of the electronic device. In some examples, an electronic device determines the temperature of a user based on temperature information corresponding to the activation of a thermal emitter (e.g., first temperature information) and temperature information corresponding to deactivation of the thermal emitter (e.g., second temperature information). In some examples, an electronic device determines a correction factor which accounts for the contact condition of the electronic device with the user based on the detected first temperature information. In some examples, the electronic device determines the correction factor by extrapolating temperature information corresponding to the temperature rise detected in the first temperature information to detect a steady-state temperature due to the activation of the thermal emitter. The systems and processes disclosed herein may reduce system errors and user errors in interaction with the electronic device; thus, improving user interaction with the electronic device.

### BRIEF DESCRIPTION OF THE DRAWINGS

FIG. 1 illustrates an electronic device that utilizes temperature data of a temperature sensing system according to some examples of the disclosure.

FIG. 2 illustrates a block diagram of an electronic device that utilizes temperature data of a temperature sensing system according to some examples of the disclosure.

FIG. 3A illustrates an electronic device worn by a user and thermal transfer therebetween according to some examples of the disclosure.

FIG. 3B illustrates a graph showing the thermal response when a thermal emitter is in an activated state and a deactivated state according to some examples of the disclosure.

FIG. 4A illustrates a graph showing a thermal response corresponding to activation of the thermal emitter according to some examples of the disclosure.

FIG. 4B illustrates a graph showing the thermal response of the detected corresponding to activation of the thermal emitter and a predicted steady-state condition according to some examples of the disclosure.

FIG. 5 illustrates a flowchart for a method of correcting a calculated temperature accounting for the contact condition between the user and the electronic device according to some examples of the disclosure.

FIG. 6 illustrates a plot of calculated temperature under different contact conditions, with and without correction for the contact condition according to some examples of the disclosure.

### DETAILED DESCRIPTION

In the following description of examples, reference is made to the accompanying drawings which form a part hereof, and in which it is shown by way of illustration specific examples that can be practiced. It is to be understood that other examples can be used, and structural changes can be made without departing from the scope of the disclosed examples.

This relates generally to systems and processes (e.g., algorithms, applications, and devices) that use temperature information for detecting the contact condition of a user-worn electronic device. Additionally or alternatively, this relates generally to systems and processes that use temperature information for further determining the temperature of a user using one or more temperature sensors of the electronic device. In some examples, an electronic device determines the temperature of a user based on temperature information corresponding to the activation of a thermal emitter (e.g., first temperature information) and temperature information corresponding to deactivation of the thermal emitter (e.g., second temperature information). In some examples, an electronic device determines a correction factor which accounts for the contact condition of the electronic device with the user based on the detected first temperature information. In some examples, the electronic device determines the correction factor by extrapolating temperature information corresponding to the temperature rise detected in the first temperature information to detect a steady-state temperature due to the activation of the thermal emitter. The systems and processes disclosed herein may reduce system errors and user errors in interaction with the electronic device; thus, improving user interaction with the electronic device.

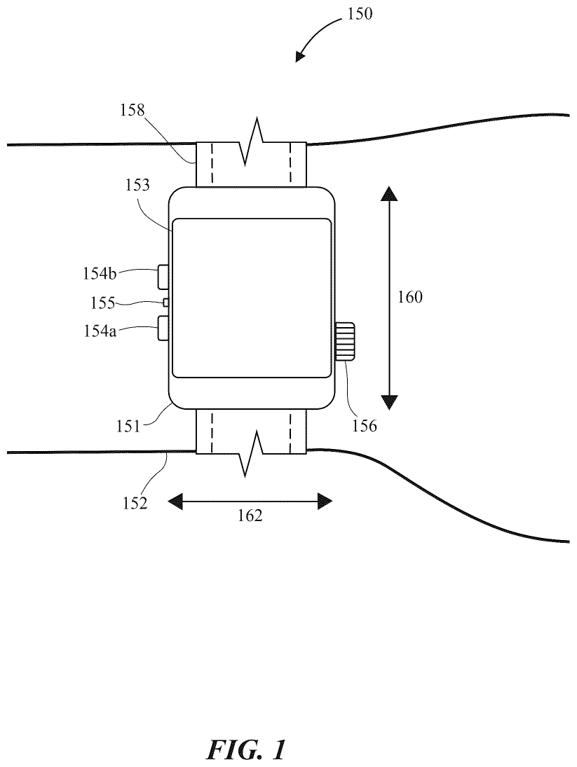

FIG. 1 illustrates an electronic device 150 (e.g., a watch, a wearable device) that utilizes temperature data of a temperature sensing system according to some examples of the disclosure. Electronic device 150 includes a housing 151 (that includes a longitudinal length along axis 160 and a width along axis 162), a touch screen 153, side buttons 154a and 154b that are optionally pressable, a speaker 155 for generating audio, and a digital crown 156 that is optionally rotatable (e.g., about an axis parallel to axis 162) and/or pressable. In some examples, the electronic device includes more or fewer buttons than in the illustrated example. Electronic device 150 is worn on (e.g., in contact with) a portion 152 (e.g., a dorsal side of a left wrist) of a user. In FIG. 1, the user wears electronic device 150 on the dorsal side of the user's left wrist and digital crown 161 faces the distal side of the user while side buttons 154a and 154b and speaker 155 faces the proximal side of the user. Electronic device 150 is coupled to a user via strap 158, or other fastener. In some examples, one or more temperature sensors distributed within electronic device 150 detect temperatures within and/or outside of electronic device 150. In some examples, the electronic device includes at least two temperature sensors to measure a difference in temperature between two locations within the electronic device. In some examples, the systems described herein can use measurements from temperature sensors to determine external temperatures (e.g., ambient air temperature, skin temperature, etc.), such as by using a model of heat flux and based on thermal resistances. Additionally, as described in more detail herein, the one or more temperature sensors can be used to determine a contact condition or correction factor to account for changes in the thermal resistance between electronic device 150 and the user due to the contact condition therebetween.

It should be understood that although illustrated and described herein primarily as a wrist worn watch, the disclosure herein is not so limited. The systems and processes described herein can be implemented in other wearable (e.g., wristband, ring, head-mounted display, glasses, etc.) and non-wearable devices (e.g., a mobile telephone, a digital media player, a personal computer, tablet computer, etc.) that optionally include a touch screen and/or optionally includes a temperature sensing system, from which temperature data is utilized in accordance with some examples of the disclosure. For example, a head-mounted display optionally includes a temperature sensing system that detects temperature data near or at temples and/or another portion of a user's forehand and utilizes the temperature data in accordance with some examples of the disclosure. For example, a non-wearable personal computer optionally includes an input surface (e.g., a keyboard, trackpad, touch-sensitive surface, laptop base, tablet, or another input surface) on which the user rests one or more portions of a user such as wrists or fingers and through which a temperature sensing system, including temperature sensors, detects temperature data corresponding to the portion of the user in contact with the input surface, and utilizes the temperature data according to some examples of the disclosure.

It should be understood that the above-described example devices, including electronic device 150, are provided by way of example, and other devices, optionally including a non-touch sensitive display, no display, or a touch-sensitive display, can include a temperature sensing system, from which temperature data is utilized in accordance with some examples of the disclosure.

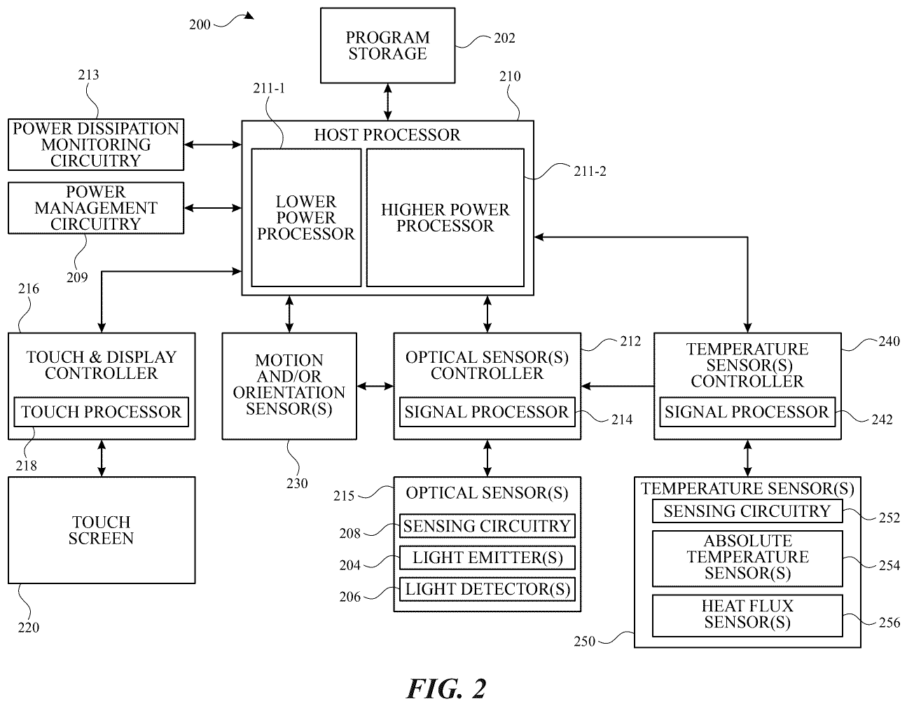

FIG. 2 illustrates a block diagram of a computing system 200 that utilizes temperature data of a temperature sensing system according to some examples of the disclosure. Computing system 200 optionally corresponds to electronic device 150 of FIG. 1 and/or may be implemented in other electronic devices, such as the different types of electronic devices discussed above.

Computing system 200 includes a host processor 210 (or more than one processor) programmed to (configured to) execute instructions and to execute operations associated with computing system 200. For example, using instructions retrieved from a program storage 202, host processor 210 can control the reception and manipulation of input and output data between components of computing system 200. Host processor 210 can be a single-chip processor (e.g., an application specific integrated circuit) or can be implemented with multiple components/circuits. For example, FIG. 2 illustrates the host processor 210 including a relatively lower power processor 211-1 and a relatively higher power processor 211-2, as described in more detail herein.

In some examples, host processor 210, together with an operating system can operate to execute computer code/programs, and produce and/or use data. The computer code and data can reside within the program storage 202 that can be operatively coupled to host processor 210.

Program storage 202 can generally provide a place to hold data used by computing system 200. Program storage 202 can be any non-transitory computer-readable storage medium. By way of example, program storage 202 can include Read-Only Memory (ROM), Random-Access Memory (RAM), hard disk drive and/or the like. The computer code and data could also reside on a removable storage medium and loaded or installed onto computing system 200 when needed. Removable storage mediums include, for example, CD-ROM, DVD-ROM, Universal Serial Bus (USB), Secure Digital (SD), Compact Flash (CF), Memory Stick, Multi-Media Card (MMC) and/or a network component.

As described herein, in some examples, host processor 210 can represent multiple processors, such as lower power processor 211-1 and higher power processor 211-2. Lower power processor 211-1 and higher power processor 211-2 can represent separate processing chips, each with independent timing and power requirements. For example, lower power processor 211-1 can operate using a first clock signal and at a first power level that allows processor 211-1 to remain operational (“on”) across most or all operating modes of computing system 200 (e.g., a sleep mode, awake mode, idle mode, etc.). By contrast, higher power processor 211-2 can operate using a second clock signal (e.g., a higher frequency clock), different from the first, or at a second power level, higher than the first. Because of the higher power requirements of higher power processor 211-2, host processor 210 (e.g., an operating system on host processor 210) can selectively disable, or power down higher power processor 211-2 or otherwise throttle its power consumption during certain operating modes of computing system 200 (e.g., a power saving mode, sleep mode, etc.). In some examples, as described herein, the higher power processor 211-1 can be powered down or otherwise throttle its power consumption to enable temperature measurements without error introduced by the power dissipation by higher power processor 211-1.

Lower power processor 211-1 and/or higher power processor 211-2 can interface with various sensors of computing system 200 including a touch sensor panel and/or a touch screen 220 (via touch and display controller 216), one or more motion and/or orientation sensors 230, one or more optical sensors 215 (via optical sensor(s) controller 212), and one or more temperature sensors 250 (via temperature sensor(s) controller 240). In some examples, lower power processor 211-1 can operate in a sleep mode or a power-saving mode, while higher power processor 211-2 is powered down. In some examples, lower power processor 211-1 can change an operating mode of computing system 200 or otherwise cause higher power processor 211-2 to be powered on (e.g., when wake up conditions are detected).

Computing system 200 can also include power management circuitry 209 and/or power dissipation monitoring circuitry 213. Host processor 210 (e.g., lower power processor 211-1 and/or higher power processor 211-2) can be coupled to power management circuitry 209 and/or power dissipation monitoring circuitry 213. Power management circuitry 209 can regulate power delivery from power supply circuitry (e.g., a battery, or another power source of computing system 200) to various components of computing system 200 (e.g., sensors, processors, antennas, displays, etc.). As an example, power management circuitry 209 can interrupt or throttle power delivery to components that generate heat within computing system 200 (e.g., heat-generating components, thermal aggressors), especially to ensure proper performance, keep the computing system in safe operating conditions, or during temperature measurements that may be sensitive to heat from such components. Power management circuitry 209 can monitor temperatures inside a housing of computing system 200 (e.g., housing 151 of FIG. 1) and/or temperatures outside the housing. In some examples, power management circuitry 209 provides control signals to inline switches coupled between the power supply circuitry of computing system 200 and various components of computing system 200, where the control signals determine an amount of current or power that can be delivered to the respective components. As an example, power management circuitry 209 can provide a first control signal to a switch interposed between a battery power source of computing system 200 and touch screen 220, such that the first control signal limits the amount of power or current delivered to the touch screen by the battery power source. As another example, power management circuitry 209 can provide a second control signal to a switch interposed between a battery power source of computing system 200 and antenna circuitry (not shown) of the system, such that the second control signal interrupts power delivery or current flow between the battery power source and the antenna circuitry.

Power dissipation monitoring circuitry 213 can monitor power supply circuitry of computing system 200, and can regulate power delivery from the power supply circuitry (not shown) to various components of computing system 200 (e.g., by sending instructions to power management circuitry 209). In some examples, power dissipation monitoring circuitry 213 includes a sensor coupled to the power supply circuitry (e.g., battery) of computing system 200. The sensor can measure power drawn by components of computing system 200 from the power supply circuitry (e.g., a battery of computing system 200). In some examples, the power drawn by components of the computing system 200 can be estimated based on a current drawn from the power supply circuitry. In some examples, the power drawn can be estimated on a device basis (e.g., estimated current drawn from the battery).

In some examples, computing system 200 includes one or more input/output (I/O) controllers that can be operatively coupled to host processor 210. I/O controllers can be configured to control interactions with one or more I/O devices (e.g., touch sensor panels, display screens, touch screens, physical buttons, dials, slider switches, joysticks, or keyboards). I/O controllers can operate by exchanging data between host processor 210 and the I/O devices that desire to communicate with host processor 210. The I/O devices and I/O controller can communicate through a data link. The data link can be a unidirectional or bidirectional link. In some cases, I/O devices can be connected to I/O controllers through wireless connections. A data link can, for example, correspond to any wired or wireless connection including, but not limited to, PS/2, Universal Serial Bus (USB), Firewire, Thunderbolt, Wireless Direct, Infra-Red (IR), Radio Frequency (RF), Wi-Fi, BLUETOOTH, or the like.

In the illustrated example, computing system 200 includes a temperature sensor(s) controller 240 operatively coupled to host processor 210 and to one or more temperature sensors 250. Also, the temperature sensor(s) controller 240 is coupled to optical sensor(s) controller 212. The one or more temperature sensors 250 include one or more absolute temperature sensors 254, one or more heat flux sensors 256, and sensing circuitry 252 (e.g., analog and/or digital circuitry to: measure signals at the one or more absolute temperature sensors 254 and/or one or more heat flux sensors 256; provide processing (e.g., amplification, filtering, level-shifting); and convert analog signals to digital signals for performing temperature and/or heat-flux sensing measurements). As an example, the one or more absolute temperature sensors 254 and one or more heat flux sensors 256 may be configured to measure temperature at different locations within the computing system 200, including at least one location or region inside the wearable device different than a location or region in which an absolute temperature sensor is disposed for the computing system 200. These temperatures and/or heat flux measurements can be used to measure temperature characteristics of the device under various modes of operation (e.g., to estimate when temperatures within a device are approaching unsafe or unsustainable levels), to estimate ambient temperatures outside the device, or to estimate a physiological signal associated with a user (e.g., a body temperature of the user). In some examples, the one or more temperatures sensors 250 include one or more absolute temperature sensors 254 without including one or more heat flux sensors 256. In some examples, the one or more temperature sensors 250 include one or more heat flux sensors 256, without including one or more absolute temperatures sensors 254.

Measured raw data from the one or more absolute temperature sensors 254, one or more heat flux sensors 256, and sensing circuitry 252 can be transferred to the host processor 210 (via temperature sensor(s) controller 240), and the host processor 210 can perform signal processing to estimate internal or external temperatures and/or to estimate physiological signals (e.g., body temperature associated with the user). Host processor 210 and/or temperature sensor(s) controller 240 can operate one or more temperature sensors 250 to measure temperature values associated with computing system 200, and to estimate temperature values associated with the environment external to the system. In some examples, temperature sensor(s) controller 240 can include signal processor 242 to sample, filter, and/or convert (from analog to digital) signals generated by one or more temperature sensors 250, which can be positioned at different locations within a housing for the computing system 200. In some examples, signal processor 242 is a digital signal processing circuit such as a digital signal processor (DSP). In some examples, the analog data measured by the one or more temperature sensors 250 can be converted into digital data by an analog to digital converter (ADC). In some examples, the digital data from the temperature sensors can be stored for processing in a buffer (e.g., a first-in-first-out (FIFO) buffer) or other volatile or non-volatile memory (not shown) in temperature sensor(s) controller 240. In some examples, host processor 210 and/or temperature sensor(s) controller 240 can store the raw data and/or processed information in memory (e.g., ROM or RAM) for historical tracking or for future diagnostic purposes. In some examples, the one or more temperature sensors 250 can include a negative temperature coefficient (NTC) temperature sensor, a resistance temperature detector (RTD), digital temperature sensor, optical temperature sensors, thin film, and/or a diode based temperature sensor.

In the illustrated example, computing system 200 includes an optical sensor(s) controller 212 operatively coupled to host processor 210 and to one or more optical sensors 215. As illustrated, in some examples, the one or more optical sensors 211 include one or more light emitters 204, one or more light detectors 206, and sensing circuitry 208 (e.g., analog circuitry to drive emitters and measure signals at the detector, provide processing (e.g., amplification, filtering), and convert analog signals to digital signals). As an example, light emitters 204 and light detectors 206 can be configured to generate and emit light into a user's skin and detect returning light (e.g., reflected and/or scattered) to measure a physiological signal (e.g., a photoplethysmogram (PPG) signal), respectively. In some examples, computing system 200 utilizes temperature data from the temperature sensing system in accordance with some examples of the disclosure to improve a PPG sensor. The absorption and/or return of light at different wavelengths can also be used to determine a characteristic of the user (e.g., oxygen saturation, heart rate) and/or about the contact condition between the one or more light emitters 204/one or more light detectors 206 and the user's skin. Measured raw data from the one or more light emitters 204, one or more light detectors 206, and sensing circuitry 208 can be transferred to host processor 210, and host processor 210 can perform the signal processing described herein to estimate a characteristic (e.g., oxygen saturation, heart rate, etc.) of the user of the example electronic device from the physiological signals. Host processor 210 and/or optical sensor(s) controller 212 can operate one or more light emitters 204, one or more light detectors 206 and/or sensing circuitry 208 to measure data from the optical sensor. In some examples, optical sensor(s) controller 212 can include timing generation for light emitters 204, light detectors 206 and/or signal processor 214 to sample, filter and/or convert (from analog to digital) signals measured from light at different wavelengths. Optical sensor(s) controller 212 can process the data in signal processor 214 and report outputs (e.g., PPG signal, relative modulation ratio, perfusion index, heart rate, on-wrist/off-wrist state, etc.) to the host processor 210. Signal processor 214 can be a digital signal processing circuit such as a digital signal processor (DSP). The analog data measured by the one or more optical sensors 215 can be converted into digital data by an analog to digital converter (ADC), and the digital data from the physiological signals can be stored for processing in a buffer (e.g., a FIFO) or other volatile or non-volatile memory (not shown) in optical sensor(s) controller 212. In some examples, some light emitters and/or light detectors are in an activated state, while other light emitters and/or light detectors are in a deactivated state (as controlled by power management circuitry 209) to conserve power, for example, or for time-multiplexing (e.g., to avoid interference between channels). In some examples, host processor 210 and/or optical sensor(s) controller 212 can store the raw data and/or processed information in memory (e.g., ROM or RAM) for historical tracking or for future diagnostic purposes.

In the illustrated example, computing system 200 includes one or more motion and/or orientation sensors 230. The one or more motion and/or orientation sensors 230 optionally includes an accelerometer (e.g., a multi-channel accelerometer (e.g., a 3-axis accelerometer), a gyroscope, and/or an inertia-measurement unit (IMU)).

In the illustrated example, computing system 200 includes a touch and display controller 216 operatively coupled to host processor 210 and to touch screen 220. Touch screen 220 can be configured to display visual output in a graphical user interface (GUI), for example. The visual output can include text, graphics, video, and any combination thereof. In some examples, the visual output can include a text or graphical representation of the physiological signal (e.g., a PPG waveform) or a characteristic of the physiological signal (e.g., oxygen saturation, heart rate, temperature, etc.) Touch screen can be any type of display including a liquid crystal display (LCD), a light emitting polymer display (LPD), an electroluminescent display (ELD), a field emission display (FED), a light emitting diode (LED) display, an organic light emitting diode (OLED) display, or the like. Host processor 210 can send raw display data to touch and display controller 216, and touch and display controller 216 can send signals to touch screen 220. Data can include voltage levels for a plurality of display pixels in touch screen 220 to project an image. In some examples, host processor 210 can be configured to process the raw data and send the signals to touch screen 220 directly. Touch and display controller 216 can also detect and track touches or near touches (and any movement or release of the touch) on touch screen 220. For example, touch processor 218 can process data representative of touch or near touches on touch screen 220 (e.g., location and magnitude) and identify touch or proximity gestures (e.g., tap, double tap, swipe, pinch, reverse-pinch, etc.). Host processor 210 can convert the detected touch input/gestures into interaction with graphical objects, such as one or more user-interface objects, displayed on touch screen 220 or perform other functions (e.g., to initiate a wake of the device or power on one or more components).

In some examples, touch and display controller 216 can be configured to send raw touch data to host processor 210, and host processor 210 can process the raw touch data. In some examples, touch and display controller 216 can process raw touch data via touch processor 218. The processed touch data (touch input) can be transferred from touch processor 218 to host processor 210 to perform the function corresponding to the touch input. In some examples, a separate touch sensor panel and display screen can be used, rather than a touch screen, with corresponding touch controller and display controller.

In some examples, the touch sensing of touch screen 220 can be provided by capacitive touch sensing circuitry (e.g., based on mutual capacitance and/or self-capacitance). For example, touch screen 220 can include touch electrodes arranged as a matrix of small, individual plates of conductive material or as drive lines and sense lines, or in another pattern. The electrodes can be formed from a transparent conductive medium such as Indium Tin Oxide (ITO) or Antimony Tin Oxide (ATO), although other partially or fully transparent and non-transparent materials (e.g., copper) can also be used. In some examples, the electrodes can be formed from other materials including conductive polymers, metal mesh, graphene, nanowires (e.g., silver nanowires) or nanotubes (e.g., carbon nanotubes). The electrodes can be configurable for mutual capacitance or self-capacitance sensing or a combination of mutual and self-capacitance sensing. For example, in one mode of operation, electrodes can be configured to sense mutual capacitance between electrodes; in a different mode of operation, electrodes can be configured to sense self-capacitance of electrodes. During self-capacitance operation, a touch electrode can be stimulated with an alternating current (AC) waveform, and the self-capacitance to ground of the touch electrode can be measured. As an object approaches the touch electrode, the self-capacitance to ground of the touch electrode can change (e.g., increase). This change in the self-capacitance of the touch electrode can be detected and measured by the touch sensing system to determine the positions of one or more objects when they touch, or come in proximity to without touching, the touch screen. During mutual capacitance operation, a first touch electrode can be stimulated with an AC waveform, and the mutual capacitance between the first touch electrode and a second touch electrode can be measured. As an object approaches the overlapping or adjacent region of the first and second touch electrodes, the mutual capacitance therebetween can change (e.g., decrease). This change in the mutual capacitance can be detected and measured by the touch sensing system to determine the positions of one or more objects when they touch, or come in proximity to without touching, the touch screen. In some examples, some of the electrodes can be configured to sense mutual capacitance therebetween and some of the electrodes can be configured to sense self-capacitance thereof.

The firmware can also be stored and/or transported within any non-transitory computer-readable storage medium for use by or in connection with an instruction execution system, apparatus, or device, such as a computer-based system, processor-containing system, or other system that can fetch the instructions from the instruction execution system, apparatus, or device and execute the instructions. In the context of this disclosure, a “non-transitory computer-readable storage medium” can be any medium (excluding signals) that can contain or store the program for use by or in connection with the instruction execution system, apparatus, or device. The computer-readable storage medium can include, but is not limited to, an electronic, magnetic, optical, electromagnetic, infrared, or semiconductor system, apparatus or device, a portable computer diskette (magnetic), a random access memory (RAM) (magnetic), a read-only memory (ROM) (magnetic), an erasable programmable read-only memory (EPROM) (magnetic), a portable optical disc such a CD, CD-R, CD-RW, DVD, DVD-R, or DVD-RW, or flash memory such as compact flash cards, secured digital cards, USB memory devices, memory sticks, and the like.

The firmware can also be propagated within any transport medium for use by or in connection with an instruction execution system, apparatus, or device, such as a computer-based system, processor-containing system, or other system that can fetch the instructions from the instruction execution system, apparatus, or device and execute the instructions. In the context of this document, a “transport medium” can be any medium that can communicate, propagate or transport the program for use by or in connection with the instruction execution system, apparatus, or device. The transport medium can include, but is not limited to, an electronic, magnetic, optical, electromagnetic or infrared wired or wireless propagation medium.

In some examples, data from one or more components of computing system 200 (e.g., touch and display controller 216, optical sensor(s) 215, motion and/or orientation sensors) are utilized along with temperature data from the one or more temperature sensors 250 according to some examples of the disclosure. In some examples, an electronic device includes more, fewer, or different components than computing system 200 and utilizes temperature data of a temperature sensing system according to some examples of the disclosure.

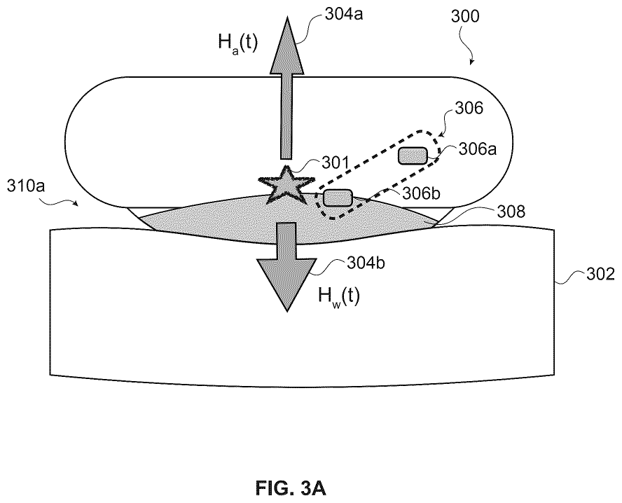

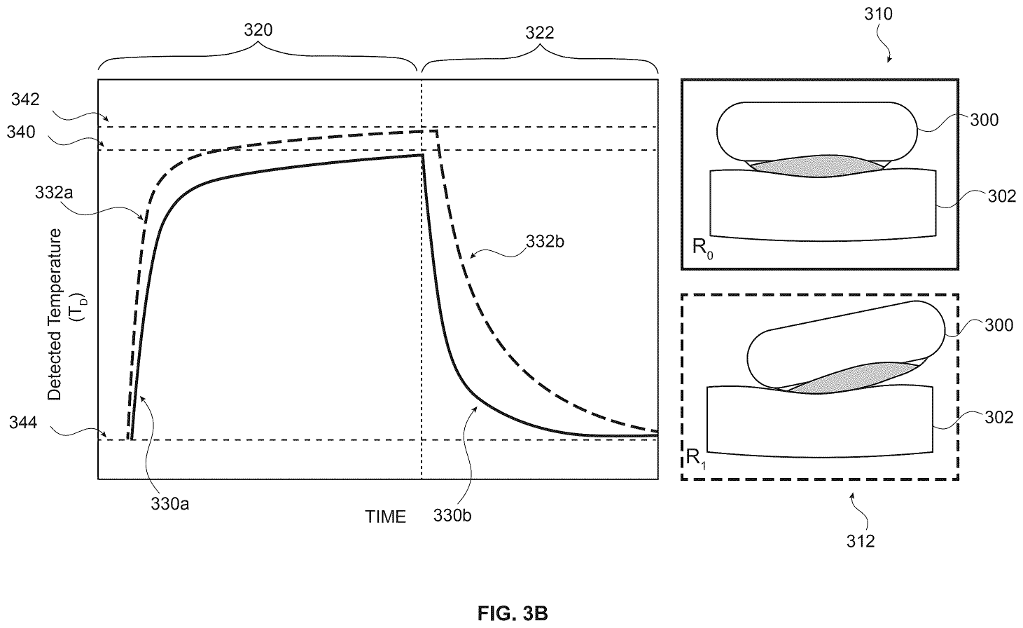

In some examples, as illustrated in FIG. 3A, the electronic device 300 is configured to be worn at the wrist of a user 302). In some examples, an electronic device 300 comprises one or more temperature sensors (e.g., 306a, and 306b, collectively referred to herein as temperature sensors 306) configured to detect the temperature at one or more locations of the electronic device 300. The temperatures measured by the one or more temperature sensors 306 can be used to estimate a temperature (e.g., surface temperature (e.g., skin temperature) and/or core temperature of the user 302). In some examples, the temperature sensors 306 comprise a first temperature sensor 306a and a second temperature sensor 306b communicatively coupled to the electronic device. In some examples, the temperature sensors are integrated within the electronic device which is configured to be worn by the user 302. In some examples, the temperature sensors are offset from an outer surface of the electronic device, wherein the outer surface is configured to interface with a surface of the user. Additionally or alternatively, the temperature sensors 306 are in contact with an outer surface (e.g., back face 308) optionally configured to interface directly with the with a surface of the user (e.g., at the wrist) when the electronic device is worn by the user. The temperature sensors are optionally communicatively connected with processing circuitry of the electronic device, wherein the processing circuitry is configured to receive temperature information detected by the one or more temperature sensors 306. In some examples, determining the temperature of the user 302 (or of the environment) is based on the heat transfer 304a, represented by label Hₐ(t), between the electronic device 300 the environment, and the heat transfer 304b, represented by H_{w}(t), between the electronic device and the user 302. For example, heat flux can be modeled to enable an estimate of a temperature outside of the device or of a user using temperatures measured by temperature sensors inside the device as described in U.S. patent application Ser. No. 17/930,041 (“the '041 application”), filed Sep. 6, 2022, which is incorporated herein by reference for all purposes. In some examples, a linear circuit model of the device can be used enable an estimate of a temperature outside of the device or of a user using temperatures measured by temperature sensors inside the device as described in the '041 application. It is understood that these model are non-limiting examples. The models can include thermal resistance parameters, including a thermal resistance between the electronic device 300 and user 302. The thermal resistance between the electronic device 300 and the user can be a function of a contact condition between electronic device 300 and user 302. Due to the nature of some wearable electronic devices, the contact condition between the electronic device and the user varies with many variables including, but not limited to: the preferences of the user (e.g., tightness of the wrist band), the perspiration of the user, the size of the electronic device, the shape of the electronic device, the materials of the electronic device, the size of the user (e.g., wrist perimeter), an activity of the user, and/or the ambient temperature.

Errors in the estimation of the temperature of the user 302 can manifest when the contact condition of the electronic device 300 with the user 302 causes a thermal resistance different than the thermal resistance parameter used to estimate the temperature of the user. For instance, when the electronic device 300 (e.g., a watch), as shown in FIG. 3A-FIG. 3B, is affixed to the user (e.g., worn) in a first contact condition 310 (or first configuration), wherein the electronic device is tightly affixed to the user and the back face 308 of the electronic device is in direct and full contact with the user 302, the thermal resistance between the electronic device and the user is a first value (R₀). When the electronic device 300 is affixed the user in a second contact condition 312 (or second configuration), wherein the electronic device is loosely in contact and/or the back face 308 of the electronic device is not in direct and/or full contact with the user 302, the thermal resistance is a second value (R₁), wherein R₁ is different than R₀. In some examples, R₁ is greater than R₀, and, in some examples, R₁ is less than R₀. In some examples, determining the thermal resistance between the electronic device 300 and the user 302 and/or of the contact condition between the electronic device 300 and the user 302, and allows for temperature sensing to be more robust and account for the contact condition/thermal resistance. As such, the errors in temperature of the user, which can be induced by inconsistent and/or non-ideal contact conditions, can be avoided, mitigated, and/or corrected. Additionally or alternatively, determining a temperature differential between the temperature detected at the first temperature sensor 306a at a first location within the electronic device 300, and the temperature detected at the second temperature sensor 306b at a second location within the electronic device, optionally allows for the identification of the contact condition and/or thermal resistance between the electronic device 300 and the user 302. Additionally or alternatively, determining the temperature response (e.g., increase or decrease) at the one or more temperature sensors 306 in response to the activation of one or more thermal emitters, and determining the contact condition of the electronic device with the user based thereon, optionally allows the electronic device to determine when the electronic device is worn by the user. The electronic device can forgo such calculations when the electronic device is not being worn by the user.

In some examples, the contact condition and/or thermal resistance between the electronic device and the user is qualified and/or quantified in relation to the interface between the electronic device and the user. In some examples, the contact condition is binary; the contact condition is a first contact condition (a “good contact” condition) or a second contact condition (a “poor contact” condition). In some examples, the contact condition can be non-binary and include additional contact conditions (e.g., good, moderate, bad, etc.). In some examples, the contact condition of the electronic device is considered to be the first contact condition when the electronic device is firmly affixed to a surface of the user (e.g., at the wrist) and/or a back-surface of the electronic device is fully in contact with the surface of the user. In some examples, the contact condition of the electronic device is considered to be the second contact condition when the electronic device is loosely affixed to a surface of the user (e.g., at the wrist) and/or a back-surface of the electronic device is partially or not in contact with the surface of the user. In some examples, the first contact condition and the second contact condition are quantified with state values, such as a value of 1 for the first contact condition and zero for the second contact condition, though it is understood that these values are non-limiting representations. In some examples, the contact condition is determined based, at least in part, on the percentage of the electronic device in physical contact with the user, wherein the percentage of the contact with the user corresponds to the quantification of the contact condition. For instance, when 100% (or a lower threshold amount such as 95%, 90%, etc.) of the back-face of a watch is in contact with the user, the contact condition of the watch with the user is equal to 1, when 0% (or an upper threshold amount such as 5%, 10%, 15%, etc.) of the back-face of a watch is in contact with the user, the contact condition of the watch with the user is equal to 0, and when a percentage is therebetween of the back-face of the watch is in contact with the user, the contact condition of the watch with the user is a value between 0 and 1. In some examples, the contact condition of the electronic device corresponds to (e.g., correlates with) the thermal resistance between the electronic device and the user. In some examples, when the electronic device is in the first contact condition, a correction factor is optionally unnecessary, and when the electronic device is in a second contact condition, the correction factor allows the electronic device to more accurately estimate the temperature of the user based on the collected and/or calculated temperature information.

In some examples, the electronic device is configured to determine the contact condition or thermal resistance of the electronic device with the user based on first temperature information. For example, the first temperature information is measured using at least one of the one or more temperature sensors 306. In some examples, temperature sensor 306b, which is relatively closer to one or more thermal emitters 301 (e.g., light emitting diodes) of the electronic device. When the first temperature information is detected, the first temperature information optionally includes an increase of temperature at the location of the one or more temperature sensors due to the activation of the one or more thermal emitters 301.

For example, as shown in FIG. 3B, the temperature profile at a temperature sensor of the electronic device varies based on factors including the contact condition and the activation of the thermal emitters. While the electronic device 300 is in a first contact condition 310, associated with thermal resistance value R₀, activation of the one or more thermal emitters 301 (at window 320) results in a first thermal response having a first profile 330a (e.g., the solid line). The first profile 330a shows the detected temperature increases over time. Optionally, the first profile 330a shows the detected temperature approaches a first steady-state temperature 340 when the one or more emitters remain activated long enough for a steady-state settling. While still in the first contact condition 310, deactivation of the one or more thermal emitters 301(at the onset of window 322) results in a second thermal response, different than the first thermal response, having a second profile 330b (e.g., the solid line). The second profile 330b shows the detected temperature decreases over time and, while the one or more emitters remain in a deactivated state, approaches second steady-state temperature 344 which corresponds to a baseline temperature.

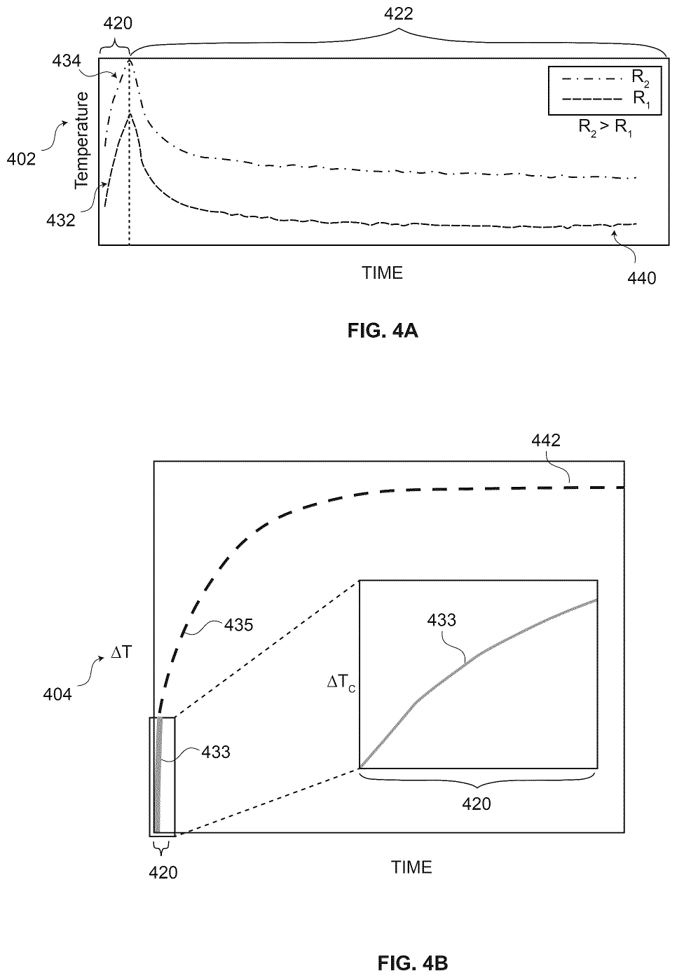

A different temperature response occurs under different contact conditions. As shown in FIG. 3B, while the electronic device is a second contact condition 312, associated with thermal resistance value R₁, activation of the one or more thermal emitters 301 (at window 320) results in a third thermal response—different than the first thermal response and the second thermal response—having a third profile 332a (e.g., the dashed line). The third profile 332a shows the detected temperature increases over time. Optionally, the third profile 332a shows the detected temperature approaches a third steady-state temperature 342 when the one or more emitters remain activated long enough for a steady-state settling. The third steady-state temperature 342 is different than the first steady-state temperature 340 and the second steady-state temperature 344 (e.g., baseline temperature). While still in the second contact condition 312, deactivation of the one or more thermal emitters 301 (at the onset of window 322) results in a fourth thermal response—different than the first thermal response, the second thermal response, and the third thermal response—having a fourth profile 332b. The fourth profile 332b shows the detected temperature decreases over time and, while the emitters remain in a deactivated state, approaches the second steady-state temperature 344 (e.g., baseline temperature). As described in more detail here, the different thermal responses based on contact condition illustrated in FIG. 3B can be leveraged to determine the contact condition. Although FIG. 3B shows thermal responses that approach and/or settle at first steady-state temperature 340 or third steady-state temperature 342, respectively, in practice the activation of the thermal emitters may not continue long enough to achieve steady-state. As described further with respect to FIG. 4B, a portion of the thermal response can be processed to predict a predicted steady-state response.

Although primarily described as using temperature sensing to determine a contact condition, the contact condition can be determined in multiple ways. For example, additionally or alternatively, in some examples, the contact condition of the electronic device with the user is optionally determined based on the absorption and/or return of light at different wavelengths in relation to the user (e.g., using optical sensor 215). Additionally or alternatively, in some examples, the contact condition between the electronic device and the user in optionally determined using proximity sensing (e.g., using capacitance, electrical resistance, ultrasonics, etc.) between the electronic device and the user, and/or using force sensing associated with the fastening of the electronic device to the user (e.g., watch band tension, using touch or force sensors on the back-surface of the electronic device).

As described herein, the different contact conditions can also result in different measurements of temperature. For example, FIG. 4A illustrates a graph showing temperature measurements corresponding to a thermal response under different contact conditions corresponding to activation of the thermal emitter according to some examples of the disclosure. Specifically, FIG. 4A illustrates the temperature 402 detected by the temperature sensing system. Thermal response 432 and thermal response 434 correspond to the temperature of the user detected under different poor contact conditions, each representing spacing between a portion of the electronic device. Thermal response 432 (e.g., dashed line) can correspond to a first spacing (and a second thermal resistance, R₁, between the electronic device and user) and thermal response 434 (e.g., dash-dotted line) can correspond to a second spacing, that is less than the first spacing (and a third thermal resistance, R₂, between the electronic device and user, more than the first thermal resistance). As a result of the relative difference spacing and different thermal resistances, as illustrated in FIG. 4A, a reduction in contact quality between the electronic device and the user results in an increase of thermal resistance, which can cause an underestimate of body temperature compared with improved contact quality. As shown in FIG. 4A, when the thermal resistance is a second value (R₁), the detected thermal response 432 to the activation of the one or more thermal emitters is a first profile, and when the thermal resistance is a third value (R₂), greater than R₁, the detected thermal response 434 to the activation of one or more thermal emitters is a second profile, wherein the detected temperatures associated with the higher thermal resistance R₂ are different (e.g., higher) than those detected in association with the lower thermal resistance R₁. Both thermal responses 432, however, show the increase in temperature due to the thermal emitter operation and the return to a baseline after the thermal emitters cease operation.

In some examples, the activation time period 420 of the one or more thermal emitters does not correspond with a period of time within which a steady-state temperature can be reached. For instance, when the one or more thermal emitters is activated for an activation time period 420 (e.g., 5 seconds, 10 seconds, 20 seconds, 40 seconds, or 60 seconds) the detected thermal response 432 (FIG. 4B) corresponds to an increase of the temperature detected at the one or more temperature sensors. In some examples, the activation time period 420 is followed by a non-activation time period 422 corresponding to a period of time during which the one or more thermal emitters are not activated. However, in order for the detected temperature to approach within a threshold of error (e.g., 5%, 10%, 20%) of a steady-state temperature (e.g., third temperature information 442), the one or more thermal emitters must be activated for a second period of time, which is longer than the first period of time (e.g., 5 mins, 10 mins, 20 mins, 30 mins, etc.). Operation of the thermal emitters for this longer period of time, however, consumes power and ties up the thermal emitters (e.g., light emitting diodes, processors, etc.) from being used for other purposes. In some examples, to conserve power and/or free up the thermal emitters, the determination of the contact condition and/or a correction factor can be achieved without achieving steady state. Instead, a steady-state temperature corresponding to the activation of the one or more thermal emitters for the second period of time can be calculated based on the temperature data while the thermal emitters are activated, such as corresponding to a temperature change 404 (ΔT) as shown in FIG. 4B. Based on the first temperature information 433, corresponding the detected thermal response 432 (at FIG. 4A) recorded during the activation of the one or more emitters, the electronic device optionally determines third temperature information 442. In some examples, the system uses curve fitting techniques to the first temperature information to generate a predicted temperature profile 435. From the temperature profile 435, the system can extrapolate to a steady-state temperature (e.g., third temperature information 442). For example, the steady-state temperature can be an asymptotic temperature that would be achieved if the one or more thermal emitters were activated until a steady-state temperature of the electronic device is reached. Different curve-fitting techniques can be used, such as polynomial regression, geometric fitting, etc., among other curve-fitting techniques.

In some examples, the third temperature information and baseline temperature information are used to compute a thermal resistance adjustment parameter (also referred to herein as a correction factor) that corresponds to the contact condition. The thermal resistance adjustment parameter can be the absolute difference between the third temperature information and the baseline temperature information, with the absolute difference divided by the power drawn by the thermal emitter during the activation time period 420 (e.g., units of temperature divided by power). The thermal resistance adjustment parameter at the contact condition can be combined (e.g., summed) with the nominal thermal resistance, which can be characterized empirically and/or can be the absolute difference between the third temperature information and the baseline temperature information with a nominal contact condition (e.g., good contact condition), with the absolute difference divided by the power drawn by the thermal emitter during the activation period. The combination of the thermal resistance adjustment parameter and the nominal thermal resistance can be used to determine an effective thermal resistance between the device and the user for use in determining body temperature.

In some examples, to support determining the thermal resistance adjustment parameter, the electronic device is configured to determine a baseline temperature of the electronic device via the one or more temperature sensing devices. The baseline temperature is optionally detected while the thermal emitters are not activated. The baseline temperature is optionally determined based on the detection of temperature information prior to the activation of the one or more thermal emitters. In some examples, the baseline temperature is optionally detected after the activation of the one or more thermal emitters ends (e.g., and the temperature returns to baseline 440 shown in FIG. 4A). In some examples, the baseline temperature is determined based on a plurality of detections of the temperature information over time. The information corresponding to the plurality of detection can be aggregated (e.g., averaged, weighted averaged, and/or corrected) to determine a baseline temperature. In some examples, the baseline temperature is based on a single instance of detecting the temperature information.

In some examples, when the electronic device activates the thermal emitters for a first process, the electronic device optionally detects the effect of heat output of the one or more thermal emitters on the electronic device and/or the user for a second process which is unrelated to the first process. For instance, in some examples, the one or more thermal emitters is activated for the purposes of detecting the pulse and/or heartrate of a user, unrelated to detecting the temperature of the user. When the one or more thermal emitters is activated for the detection of the pulse and/or heartrate of the user, the electronic device optionally records first temperature information while the one or more thermal emitters is activated. In some examples, the first temperature information corresponds to a transient response of the temperature sensors of the electronic device. For instance, when the one or more thermal emitters corresponds to an LED (or multiple LEDs), the activation of the LED or LEDs results in an increase of temperature over the window 320, during the activation time period 420 of the LED. The amount of power associated with the activation of the one or more thermal emitters optionally corresponds, at least in-part, to the transient response of the electronic device and/or the portion of the user which the electronic device is interfacing with.

In some examples, the electronic device detects the heat transfer and/or thermal response between the electronic device and the user by detecting the change in temperature detected at the one or more temperature sensors 306 in response to activation of the one or more thermal emitters 301. The thermal emitters (e.g., LEDs) are optionally used for alternate functions such as related to detecting of physiological characteristics (e.g., oxygen saturation, heart rate, etc.) and are optionally activated on a periodic basis as required for operations unrelated to temperature sensing. In some examples, the electronic device opportunistically takes advantage of the periodic activation of thermal emitters for non-temperature sensing operations to detect temperature data, determine a correction factor based on the temperature data, and/or apply the correction factor to the temperature data to provide a more accurate determination of the temperature of the user. By opportunistically taking advantage of the activation of the thermal emitters, the electronic device is able to detect the first temperature information required for correcting the temperature of the user without independently expending power (e.g., battery power) to detect the first temperature information.

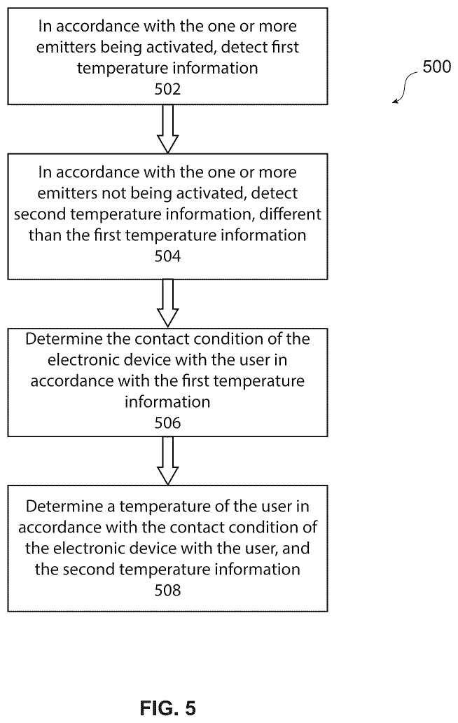

In some examples, the electronic device is configured to perform a method 500 as shown in FIG. 5. Method 500 includes temperature measurement to determine contact condition, and temperature measurement to determine a temperature of the user, which optionally accounts for the contact condition. Method 500 includes, in accordance with the one or more thermal emitters being activated, detecting a first temperature information (at 502), such as using temperature sensor 306b. Method 500 also includes determining the contact condition of the electronic device with the user in accordance with the first temperature information (at 506). Additionally, method 500 includes, in accordance with the one or more thermal emitters 301 not being activated (e.g., to avoid the noise aggressor of the thermal emitters), detecting second temperature information (e.g., measurements from temperature sensor 306a and 306b), different than the first temperature information (at 504). The method includes determining a temperature of the user based on the second temperature information (at 508), such as using a temperature model or a function derived from a temperature model. The temperature of the user is also determined in accordance with the contact condition of the electronic device with the user (e.g., to mitigate errors that would otherwise be introduced by a poor contact condition).

In some examples, the electronic device is configured to detect the first temperature information while activating the thermal emitters (at 502). In some examples, the electronic device detects the first information while the thermal emitters are activated, the electronic device is configured to use the collected first information for use in determining the contact condition (or a correction factor) when the activation time of the thermal emitters is at least a time threshold. In some examples, activation of the time of the thermal emitters is at least the time threshold, the thermal emitters can cause sufficient heat generation within the electronic device to generate a thermal response that can be used to generate third information. For example, when the thermal emitters are not activated for at least the time threshold, the thermal response curve corresponding to the first temperature information (e.g., first temperature information 433) is not enough data to accurately estimate the third temperature information 442. Implementors will understand that the time threshold is optionally a function of the thermal characteristics of the thermal emitters and the thermal characteristics of the electronic device, among other factors. In some examples, the time threshold is 1 second, 5 seconds, 10 seconds, 30 seconds, or 1 minute. In some examples, the electronic device optionally leverages operation of the thermal emitters for other purposes to opportunistically measure the first temperature information. For example, the electronic device optionally uses a thermal emitter, such as one or more optical sensors, to determine a physiological characteristic of the user (e.g., PPG, heart rate, blood oxygenation, etc.) or to determine whether the electronic device is worn by the user (e.g., on-wrist versus off-wrist). Using the first information collected while the one or more thermal emitters are activated longer than the activation time threshold, provides a higher fidelity of data for the calculation of the third temperature information. When the detected first temperature information does not correspond to the thermal emitters being activated for more than the activation time threshold, the electronic device optionally forgoes using the collected first information and optionally forgoes calculation of the third temperature information until first temperature information is collected while the thermal emitters are activated for a period long enough to satisfy the time threshold.

In some examples, the electronic device performs the processing of the first temperature information (e.g., to determine the contact condition) each time the first temperature information is acquired. In some examples, to reduce power consumption, the electronic device collects and/or processes the first temperature information after a time threshold (“a detection interval threshold”) passes from the previous processing. For example, detecting the first temperature information and the resulting determination of the contact condition within a detection interval threshold may be unnecessary, and result in similar results to those gained by using previously obtained first temperature information. In some examples, when the electronic device determines that the first temperature information has not been detected in more than detection interval time threshold (e.g., 5 minutes, 10 minutes, 20 minutes, 30 minutes, 60 minutes, or more than 60 minutes), the electronic device detects and processes the first temperature information in accordance with the activation of the one or more thermal emitters. Additionally or alternatively, when the electronic device determines that the first temperature information has been detected within the detection interval time threshold, the electronic device optionally forgoes detecting and/or processing the first temperature information. By forgoing detecting and/or processing the first temperature information when the first temperature information was previously detected within the detection interval time threshold, the electronic device mitigates unnecessary detections and/or processes to reduce power consumption and/or conserve battery life.

In some examples, when the thermal emitters are in a deactivated state (not activated, and optionally with a buffer to allow for settling post-activation), the electronic device detects and/or records second temperature information (at 504), via the one or more temperature sensors. In some examples, the second temperature information can correspond to a baseline temperature (e.g., used to determine the correction factor as described above), which is approached when the electronic device and the user reach a steady-state condition while the one or more thermal emitters is not activated. The baseline temperature optionally provides a reference temperature which corresponds to a steady-state temperature of a temperature sensor of the electronic device while the electronic device is being worn and the thermal emitters are not activated. Additionally or alternatively, in some examples, the second temperature information includes detected temperatures at the one or more emitters, which can be used for determining a temperature of the user.

In some examples, the baseline temperature information is detected prior to the activation of the one or more thermal emitters to prevent the artificial increase of the second temperature information. In some examples, the second temperature information is detected after a non-activation time threshold corresponding to the time since the previous activation of the one or more thermal emitters. For instance, in some examples, the non-activation time threshold corresponding to the time since the previous activation of the one or more thermal emitters optionally corresponds to 1 minute, 5 minutes, 10 minutes, 20 minutes, or 25 minutes. Detecting the second temperature when the non-activation time threshold has not been satisfied (e.g., earlier) would potentially result in potential lingering effects of the activation of the thermal emitters and/or the baseline temperature information being affected by the activation of the thermal emitters.

In some examples, to obtain an accurate baseline, the electronic device is worn for at least a time threshold, which corresponds to an amount of time within which the electronic device reaches or approaches (e.g., within 1%, within 5%, etc., of) a thermal equilibrium between the electronic device and the user while the one or more thermal emitters are not activated. A steady-state time threshold optionally corresponds to 1 minute, 5 minutes, or 10 minutes, for example. In some examples, a steady-state condition corresponds to an equilibrium or near equilibrium with respect to heat transfer between the electronic device and the user. In some examples, environmental variables (e.g., ambient temperature, electronic device operations, activation of thermal emitters within the electronic device, change of environment of the user (e.g., indoors/outdoors), solar heat gain, and/or air-flow), and/or contact condition between the electronic device and the user effect the baseline temperature. In some examples, one or more environmental variables are accounted for in the calculation of the correction factor, and/or the calculation of the temperature of the user.

In some examples, the electronic device determines the contact condition of the electronic device with the user (at 506), in order to account for variability of temperature information due to the contact condition of the electronic device with the user. In some examples, the electronic device calculates a correction factor to account for varying contact conditions and/or environmental variables. In some examples, the baseline temperature information allows the calculation of a correction factor. The correction factor is based on the temperature difference between the baseline temperature information (e.g., baseline 440) and the third temperature information (e.g., third temperature information 442). In some examples, the correction factor corresponds to, at least in part, the power output corresponding to the activation of the thermal emitter. In some examples, the correction factor corresponds to, at least in part, an amount of thermal energy produced by the one or more thermal emitters when the one or more thermal emitters is activated. For instance, when an LED (or multiple LEDs) is activated, a portion of the power required to activate the LED is translated to the illumination of the LED and a portion of the power required to activate the LED is translated into thermal energy (e.g., heat), which effects the first temperature information.

In some examples, in accordance with determining the contact condition and the correction factor, the electronic device applies the correction factor based, at least in part, on the contact condition of the electronic device with the user and/or the second temperature information (at 508) to increase the accuracy of the estimated temperature of the user. In some examples, applying the correction factor includes determine an effective thermal resistance between the device and the user for use in determining body temperature. In some examples, the effective thermal resistance is based on a combination (e.g., sum) of the thermal resistance adjustment parameter at the contact condition with the nominal thermal resistance at the nominal contact condition.

In some examples, the electronic device determines the correction factor based, at least in part, on the difference between the baseline temperature of the user, and the third temperature information corresponding to the calculated and/or predicted steady-state temperature in response to the activation of the one or more thermal emitters. Additionally or alternatively, the electronic device optionally determines the correction factor based, at least in part, on the power corresponding to the activation of the one or more thermal emitters. In some examples, the power drawn from the one or more thermal emitters 301 can be estimated based on a current drawn from the power supply circuitry connected to a power supply (e.g., electronic device battery). In some examples, the power drawn can be estimated on the power drawn by the one or more thermal emitters (e.g., estimated current drawn from the battery). In some examples, the power drawn can be estimated using power dissipation monitoring circuitry 213. In some examples, the correction factor is configured to mitigate inaccuracies related to a difference between the temperature of the electronic device and the temperature of the user, and to provide a more accurate estimation of the core and/or surface temperature of the user. In some examples, the correction factor is based, at least in part, on the contact condition of the electronic device with the user.

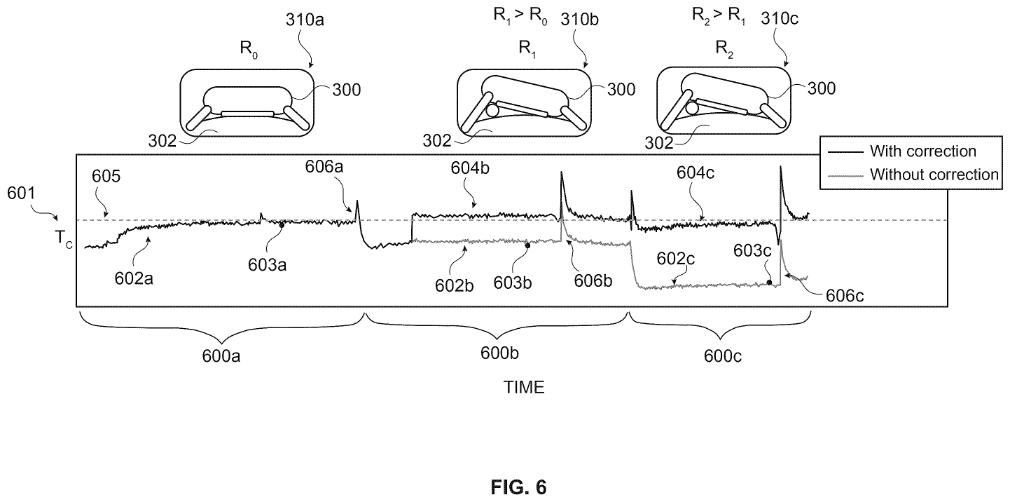

FIG. 6 illustrates a plot of calculated temperature under different contact conditions, with and without correction for the contact condition according to some examples of the disclosure. In the example of FIG. 6, the temperature (T_{c}) represents a calculated temperature 601 of the user. FIG. 6 also includes three different spikes in temperature due to activation of the thermal emitters (e.g., at 606a, 606b, and 606c). In some examples, the electronic device applies a correction factor to detected and/or calculated temperatures 601 corresponding to a temperature of the user based on the one or more temperatures detected by the one or more temperature sensors.

As shown in FIG. 6, during a first window 600a, the electronic device 300 is donned in a first contact condition 310a, with the user 302, wherein the thermal resistance is R₀ (e.g., nominal thermal resistance). After donning, the electronic device approaches a steady-state temperature with the user. In the example of FIG. 6, the calculated temperature (T_{c}) represents the body temperature of the user. As shown, the calculated temperature 602a approaches the actual temperature 605 of the user. After reaching steady-state, the baseline information 603a is optionally detected.

A temperature increase 606a corresponds with the activation of the thermal emitter at which time first temperature information is optionally detected. As shown, after reaching steady-state in the first contact condition 310a, the calculated temperature 602a approximates the actual temperature 605 of the user. As a result, in some examples, correction of the calculated temperature 601 of the user is optionally unnecessary (or it may be applied with minimal impact on the result).

At the end of first window 600a, the electronic device 300 is in a second contact condition 310b with the user 302 wherein thermal resistance is R₁, is greater than R₀. The calculated temperature 601 of the user, due to the increased thermal resistance, includes a greater error than the first contact condition as exemplified in the differences between calculated temperature 602b within a second window 600b and the actual temperature 605 of the user. The error remains without correction, but the application of a correction factor causes the calculated temperature 604b of the user to better approximate the actual temperature 605 of the user, with the exception of the activation of the emitters at 606b. Optionally the first temperature information can be collected at 606b and the new baseline temperature information at 603b, for example, for continued processing to determine the correction factor and/or contact condition.

In some examples, in third window 600c, the electronic device 300 is in a third contact condition 310c with the user 302 wherein thermal resistance is R₂, is greater than R₁. The calculated temperature 601 of the user, due to the increased thermal resistance, includes an even greater error than the second contact condition as exemplified in the differences between calculated temperature 602c without a correction factor applied within third window 600c and the actual temperature 605 of the user. Following the application of a correction factor, the calculated temperature 604c of the user better approximates the actual temperature 605 of the user. Optionally additional the first temperature information can be collected at 606c and the new baseline temperature information at 603c.

In some examples, the electronic device is configured to detect motion data from one or more multi-channel motion sensors (e.g., accelerometers) to determine motions of the electronic device corresponding to motions of the user. The motions of the electronic device allow the electronic device to determine when the electronic device is worn by the user and/or when the user is engaged in an activity (e.g., running) which may affect the contact condition of the electronic device with the user and/or the detection of the first and/or second temperature information. In some examples, when the electronic device determines that the motion data from the one or more multi-channel motion sensors exceeds a first motion threshold, the electronic device optionally forgoes detecting and/or collecting the first temperature information (and/or the baseline information) and/or the second temperature information. Additionally or alternatively, when the electronic device determines that the motion data from the one or more multi-channel motion sensors indicates that the electronic device is not worn by the user, the electronic device optionally forgoes detecting and/or collecting the temperature information. Additionally or alternatively, when the electronic device determines that the motion data from the one or more multi-channel motion sensors indicates that the electronic device is worn by the user, and the motion data is less than a first motion threshold, the electronic device optionally detects and/or collects the first and/or second temperature information. While indications of the electronic device being worn by the user are optionally provided through the detection of motion data from one or more multi-channel motion sensors, alternate methods to determines when the electronic device is worn (e.g., on-wrist detection) are within the spirit and scope of the present disclosure of the present disclosure.

Additionally or alternatively, although methods discussed herein are directed toward examples corresponding to the correction of the calculation of the temperature of the user, in some examples, the methods disclosed herein can be used to augment physiological sensing corresponding to the detection and/or determination of other physiological data including, but not limited to: Electrocardiogram (ECG), PPG, blood oxygenation levels, etc.

Therefore, according to the above, some examples of the disclosure are directed to a method. The method optionally comprises, at an electronic device comprising one or more temperature sensors, one or more emitters (e.g., thermal emitters), and processing circuitry: in accordance with the one or more emitters being activated, detect first temperature information; in accordance with each of the one or more emitters not being activated, detecting second temperature information, different than the first temperature information, determining a contact condition of the electronic device with the user in accordance with the first temperature information, and determining a temperature (e.g., core temperature, and/or surface temperature) of the user in accordance with the contact condition of the electronic device with the user, and the second temperature information.

Additionally or alternatively to the one or more examples disclosed above, in some examples, the method further comprises determining third temperature information (e.g., predictive steady-state temperature information) based at least on the first temperature information, optionally including a first temperature model corresponding to temperatures external to the user of the electronic device.

Additionally or alternatively to the one or more examples disclosed above, in some examples, determining the third temperature information includes determining a function, wherein the function is fit to the first temperature information, and extrapolating the third temperature information using the function.

Additionally or alternatively to the one or more examples disclosed above, in some examples, the first temperature information corresponds to a transient response to heating (of the user and/or the electronic device) from activation of the one or more emitters and the third temperature information corresponds to a predicted steady-state response based on the activation of the one or more emitters.

Additionally or alternatively to the one or more examples disclosed above, in some examples, the method further comprises determining the contact condition between the electronic device and the user, using the third temperature information.

Additionally or alternatively to the one or more examples disclosed above, determining the contact condition between the electronic device and the user includes determining a correction factor corresponding to a thermal resistance between the user and the electronic device.

Additionally or alternatively to the one or more examples disclosed above, in some examples, determining the temperature of the user in accordance with the contact condition of the electronic device with the user and the second temperature information comprises applying the correction factor.

Additionally or alternatively to the one or more examples disclosed above, in some examples, the contact condition of the electronic device with the user includes first contact condition or a second contact condition.

Additionally or alternatively to the one or more examples disclosed above, in some examples, the method further comprises determining a baseline temperature corresponding to the temperature of the electronic device, when the emitters are not activated.

Additionally or alternatively to the one or more examples disclosed above, determining the correction factor corresponds to determining a difference between baseline temperature of the user, and the third temperature information.

Additionally or alternatively to the one or more examples disclosed above, in some examples, the one or more emitters include one or more LEDs.

Additionally or alternatively to the one or more examples disclosed above, in some examples, the method further comprises, in accordance with a determination that the one or more emitters are activated for a period of time exceeding a time threshold measured from the activation of the one or more emitters, using the first temperature information to determine the contact condition; and in accordance with a determination that the one or more emitters are activated for a period of time below the time threshold, forgoing determining the contact condition of the electronic device with the user in accordance with the first temperature information. Additionally or alternatively, the time threshold measured from the activation of the one or more emitters is 20 seconds.

Additionally or alternatively to the one or more examples disclosed above, in some examples, the method further comprises: in accordance with a determination that one or more first criteria are satisfied, including a criterion that the first temperature information has not been detected within a time threshold measured from a last record of the first temperature information, updating the first temperature data; and in accordance with a determination that the one or more first criteria are not satisfied, forgoing detecting first temperature information.

Additionally or alternatively to the one or more examples disclosed above, in some examples, the method further comprises: detecting motion data from one or more multi-channel motion sensors; in accordance with a determination that one or more first criteria are satisfied, including a criterion that the motion data does not exceed a first movement threshold, recording the first temperature data; and in accordance with a determination that the one or more first criteria are not satisfied, forgoing detecting the first temperature information.

Additionally or alternatively to the one or more examples disclosed above, in some examples, the method further comprises detecting when a user is wearing the electronic device; in accordance with a determination that one or more first criteria are satisfied, including a criterion that the user has been wearing the electronic device for longer than a time threshold measured from a determination of when the electronic device is initially donned by the user, recording the first temperature data; and in accordance with a determination that the one or more first criteria are not satisfied, forgoing detecting first temperature information.

Some examples of the disclosure are directed to a non-transitory computer readable storage medium. The non-transitory computer readable storage medium can store instructions, which when executed by an electronic device comprising processing circuitry, can cause the processing circuitry to perform any of the above methods. Some examples of the disclosure are directed to an electronic device comprising processing circuitry, memory, and one or more programs. The one or more programs can be stored in the memory and configured to be executed by the processing circuitry. The one or more programs can include instructions for performing any of the above methods. Some examples of the disclosure are directed to an electronic device, comprising one or more processors, memory, and means for performing any of the above methods. Some examples of the disclosure are directed to an information processing apparatus for use in an electronic device, the information processing apparatus comprising means for performing any of the above methods.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates examples in which users selectively block the use of, or access to, personal information data. That is, the present disclosure contemplates that hardware and/or software elements can be provided to prevent or block access to such personal information data. For example, in the case of sharing physiological data the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed examples, the present disclosure also contemplates that the various examples can also be implemented without the need for accessing such personal information data. That is, the various examples of the present technology are not rendered inoperable due to the lack of all or a portion of such personal information data. For example, user feedback corresponding to physiological sensing can be generated by inferring preferences based on non-personal information data or a bare minimum amount of personal information, such as the content being requested by the device associated with a user, other non-personal information available to the service, or publicly available information.

The foregoing description, for purpose of explanation, has been described with reference to specific examples. However, the illustrative discussions above are not intended to be exhaustive or to limit the disclosure to the precise forms disclosed. Many modifications and variations are possible in view of the above teachings. The examples were chosen and described in order to best explain the principles of the disclosure and its practical applications, to thereby enable others skilled in the art to best use the disclosure and various described examples with various modifications as are suited to the particular use contemplated.

Although examples of this disclosure have been fully described with reference to the accompanying drawings, it is to be noted that various changes and modifications will become apparent to those skilled in the art. Such changes and modifications are to be understood as being included within the scope of examples of this disclosure as defined by the appended claims.

1. An electronic device, comprising:
one or more temperature sensors;
one or more emitters; and
processing circuitry configured to:
in accordance with at least one of the one or more emitters being in an activated state, detect first temperature information;
in accordance with each of the one or more emitters being in a deactivated state, detect second temperature information, different than the first temperature information;
determine a contact condition of the electronic device with a user in accordance with the first temperature information; and
determine a temperature of the user in accordance with the contact condition of the electronic device with the user, and the second temperature information.

2. The electronic device of claim 1, wherein the processing circuitry is further configured to:
determine third temperature information based at least on the first temperature information.

3. The electronic device of claim 1, wherein determining the contact condition between the electronic device and the user comprises determining a correction factor corresponding to a thermal resistance between the user and the electronic device.

4. The electronic device of claim 1, wherein the processing circuitry is further configured to determine a baseline temperature corresponding to the temperature of the electronic device, when the one or more emitters are in the deactivated state.

5. The electronic device of claim 1, wherein the electronic device is further configured to:
in accordance with a determination that the at least one of the one or more emitters are in the activated state for a period of time exceeding a time threshold measured from activation of the at least one of the one or more emitters, determining the contact condition using the first temperature information; and
in accordance with a determination that the at least one of the one or more emitters are in the activated state for a period of time below the time threshold, forgo determining the contact condition of the electronic device with the user in accordance with the first temperature information.

6. The electronic device of claim 1, wherein the electronic device is further configured to:
in accordance with a determination that one or more first criteria are satisfied, including a criterion that the first temperature information has not been detected within a time threshold measured from a last record of the first temperature information, update the first temperature information; and
in accordance with a determination that the one or more first criteria are not satisfied, forgo detecting the first temperature information.

7. The electronic device of claim 1, wherein the electronic device is further configured to detect motion data from one or more multi-channel motion sensors;
in accordance with a determination that one or more first criteria are satisfied, including a criterion that the motion data does not exceed a first movement threshold, record the first temperature information; and
in accordance with a determination that the one or more first criteria are not satisfied, forgo detecting first temperature information.

8. The electronic device of claim 1, wherein the electronic device is further configured to:
detect when a user is wearing the electronic device;
in accordance with a determination that one or more first criteria are satisfied, including a criterion that the user has been wearing the electronic device for longer than a time threshold measured from a determination the electronic device is initially donned by the user, record the first temperature information; and
in accordance with a determination that the one or more first criteria are not satisfied, forgo detecting first temperature information.

9. A method comprising:
at an electronic device including one or more temperature sensors, one or more emitters, and processing circuitry:
in accordance with at least one of the one or more emitters being in an activated state, detect first temperature information;
in accordance with each of the one or more emitters being in a deactivated state, detect second temperature information, different than the first temperature information;
determining a contact condition of the electronic device with a user in accordance with the first temperature information; and
determining a temperature of the user in accordance with the contact condition of the electronic device with the user, and the second temperature information.

10. The method of claim 9, further comprising:
determining third temperature information based at least on the first temperature information.

11. The method of claim 9, wherein determining the contact condition between the electronic device and the user comprises determining a correction factor corresponding to a thermal resistance between the user and the electronic device.

12. The method of claim 9, wherein the processing circuitry is further configured to determine a baseline temperature corresponding to the temperature of the electronic device, when the one or more emitters are in the deactivated state.

13. The method of claim 9, further comprising:
in accordance with a determination that the at least one of the one or more emitters are in the activated state for a period of time exceeding a time threshold measured from activation of the at least one of the one or more emitters, determining the contact condition using the first temperature information; and
in accordance with a determination that the at least one of the one or more emitters are in the activated state for a period of time below the time threshold, forgoing determining the contact condition of the electronic device with the user in accordance with the first temperature information.

14. The method of claim 9, further comprising:
in accordance with a determination that one or more first criteria are satisfied, including a criterion that the first temperature information has not been detected within a time threshold measured from a last record of the first temperature information, update the first temperature information; and
in accordance with a determination that the one or more first criteria are not satisfied, forgo detecting the first temperature information.

15. A non-transitory computer readable storage medium storing instructions, which when executed by an electronic device including one or more temperature sensors, one or more emitters, and processing circuitry, cause the processing circuitry to:
in accordance with at least one of the one or more emitters being in an activated state, detect first temperature information;
in accordance with each of the one or more emitters being in a deactivated state, detect second temperature information, different than the first temperature information;
determine a contact condition of the electronic device with a user in accordance with the first temperature information; and
determine a temperature of the user in accordance with the contact condition of the electronic device with the user, and the second temperature information.

16. The non-transitory computer readable storage medium of claim 15, wherein the instructions further cause the processing circuitry to:
determine third temperature information based at least on the first temperature information.

17. The non-transitory computer readable storage medium of claim 15, wherein determining the contact condition between the electronic device and the user comprises determining a correction factor corresponding to a thermal resistance between the user and the electronic device.

18. The non-transitory computer readable storage medium of claim 15, wherein the instructions further cause the processing circuitry to determine a baseline temperature corresponding to the temperature of the electronic device, when the one or more emitters are in the deactivated state.

19. The non-transitory computer readable storage medium of claim 15, wherein the instructions further cause the processing circuitry to:
in accordance with a determination that the at least one of the one or more emitters are in the activated state for a period of time exceeding a time threshold measured from activation of the at least one of the one or more emitters, determine the contact condition using the first temperature information; and
in accordance with a determination that the at least one of the one or more emitters are in the activated state for a period of time below the time threshold, forgo determining the contact condition of the electronic device with the user in accordance with the first temperature information.

20. The non-transitory computer readable storage medium of claim 15, wherein the instructions further cause the processing circuitry to:
in accordance with a determination that one or more first criteria are satisfied, including a criterion that the first temperature information has not been detected within a time threshold measured from a last record of the first temperature information, update the first temperature information; and
in accordance with a determination that the one or more first criteria are not satisfied, forgo detecting the first temperature information.
